(19) United States
(12) Patent Application Publication
(10) Pub. No.: US 2017/0336520 A1
(43) Pub. Date: Nov. 23, 2017

(54) RADIOGRAPHIC CAPTURING APPARATUS

(71) Applicant: Konica Minolta, Inc., Tokyo (JP)

(72) Inventor: Kazunori OKAZAWA, Tokyo (JP)

(21) Appl. No.: 15/590,463

(22) Filed: May 9, 2017

(30) Foreign Application Priority Data
May 17, 2016 (JP) 2016-098363

(51) Int. Cl. G01T 1/175; A61B 6/00; G01T 1/105; G01T 1/29

(52) U.S. Cl. CPC G01T 1/175; G01T 1/2921; A61B 6/548; G01T 1/105

(57) ABSTRACT

A radiographic capturing apparatus includes the following. A wireless communication circuit or a wireless communication device establishes wireless communication with an external unit. A power supply circuit feeds electrical power to the wireless communication circuit or the wireless communication device and is switchable between a low-load mode and a high-load mode. The power supply circuit is able to feed a current having a larger value in the high-load mode than in the low-load mode. A power supply efficiency of the power supply circuit in the high-load mode is lower than that in the low-load mode when a current having a small value as in the low-load mode is fed in the high-load mode. A switcher switches a load mode of the power supply circuit based on the detected or obtained load state. A built-in power supply feeds electrical power to the power supply circuit.

### CROSS REFERENCE TO RELATED APPLICATIONS

The present invention claims priority under 35 U.S.C. §119 to Japanese Application Patent Application No. 2016-098363 filed on May 17, 2016 the entire content of which is incorporated herein by reference.

### BACKGROUND OF THE INVENTION

### Field of the Invention

The present invention relates to a radiographic capturing apparatus and in particular to a radiographic capturing apparatus including a built-in power supply.

### Description of Related Art

Various radiographic capturing apparatuses have been developed, such as a direct radiographic capturing apparatus that generates charges in detecting elements in proportion to the doses of incident radiation and converts the charges into electrical signals and an indirect radiographic capturing apparatus that converts incident radiation into electromagnetic waves having different wavelengths, such as visible light, with a scintillator, generates charges in photoelectric transducers, such as photodiodes, in proportion to the energy of the electromagnetic waves, and converts the charges into electrical signals (i.e., image data). In the present invention, the detecting elements of a direct radiographic capturing apparatus and the photoelectric transducers of an indirect radiographic apparatus are collectively referred to as “radiation detecting elements”.

Such a radiographic capturing apparatus is known as a flat panel detector (FPD). A traditional FPD is of a dedicated type (stand-alone type) and is integrated with a platform. Recently, a portable (cassette type) radiographic capturing apparatus has been developed and put to practical use that includes a casing accommodating radiation detecting elements. Such a portable radiographic capturing apparatus (hereinafter simply referred to as “radiographic capturing apparatus”) usually includes a power supply circuit that supplies electrical power to the functional components of the apparatus, such as a controller, and a built-in power supply that supplies electrical power to the power supply circuit.

A radiographic capturing apparatus having high power consumption exhausts the built-in power supply, and causes a reduction in the operable time of the radiographic capturing apparatus per charge of the built-in power supply, frequent charging of the built-in power supply of the radiographic capturing apparatus, and/or a reduction in the number of capturable images per charge, thereby reducing the efficiency of image capturing by the radiographic capturing apparatus.

Japanese Unexamined Patent Application Publication No. 2009-206762 discloses a communication terminal that selects a reception mode that has a minimum duration of an awake state of the communication terminal through control of the number of received packets and timer values and switches the reception mode in response to every reception of a beacon from an access point at a predetermined cycle, to optimize energy consumption during reception. Such a technique may be applied to the radiographic capturing apparatus so that the power consumption of the built-in power supply of the radiographic capturing apparatus is reduced by switching the reception mode.

Unfortunately, the reduction in power consumption of the built-in power supply of the radiographic capturing apparatus cannot be readily achieved even with the technique described above. The inventors of the present invention have conducted extensive research and discovered a technique for reducing power consumption of the built-in power supply of a radiographic capturing apparatus.

### BRIEF SUMMARY OF THE INVENTION

An object of the present invention, which has been conceived to solve the drawbacks described above, is to provide a radiographic capturing apparatus including a built-in power supply that has low power consumption.

According to an aspect of the present invention, there is provided a radiographic capturing apparatus including: a two-dimensional array of a plurality of radiation detecting elements; a wireless communication circuit or a wireless communication device which establishes wireless communication with an external unit; and a power supply circuit which feeds electrical power to the wireless communication circuit or the wireless communication device and is switchable between a low-load mode and a high-load mode, the power supply circuit being able to feed a current having a larger value in the high-load mode than in the low-load mode, a power supply efficiency of the power supply circuit in the high-load mode being lower than the power supply efficiency of the power supply circuit in the low-load mode when a current having a small value as in the low-load mode is fed in the high-load mode; a switcher which detects or obtains a load state of the wireless communication circuit or the wireless communication device and switches a load mode of the power supply circuit based on the detected or obtained load state of the wireless communication circuit or wireless communication device; and a built-in power supply which feeds electrical power to the power supply circuit.

### BRIEF DESCRIPTION OF THE DRAWINGS

The present invention will become more fully understood from the detailed description given hereinbelow and the appended drawings, and thus are not intended to define the limits of the present invention, and wherein;

FIG. 1 is an external perspective view of a radiographic capturing apparatus according to an embodiment;

FIG. 2 is a block diagram illustrating an equivalent circuit of the radiographic capturing apparatus;

FIG. 3 illustrates the radiographic capturing apparatus receiving a beacon from an access point;

FIG. 4A illustrates an example variation in the load state of a wireless communication circuit or wireless communication device during transmission of a beacon;

FIG. 4B illustrates the switching of a load mode of a power supply circuit in accordance with the load state of the wireless communication circuit or wireless communication device;

FIG. 5 illustrates an example relation of a current value and power supply efficiency in a low-load mode A and a high-load mode B of the power supply circuit;

FIG. 6 illustrates transmission of signals from the wireless communication circuit or wireless communication device and a controller, and a power value sent from the power supply circuit, in the embodiment;

FIG. 7A illustrates another example variation in the load state of the wireless communication circuit or wireless communication device during transmission of a beacon;

FIG. 7B illustrates switching of the load mode of the power supply circuit in accordance with the load state of the wireless communication circuit or wireless communication device;

FIG. 8 illustrates detection of a power value sent from the power supply circuit by the controller, the transmission of a signal from the controller, and the transmission of a power value from the power supply circuit, in a first modification; and

FIG. 9 illustrates detection of a power value sent from the power supply circuit to the wireless communication circuit or wireless communication device by a current detector of the power supply circuit, in a second modification.

### DETAILED DESCRIPTION OF PREFERRED EMBODIMENTS

A radiographic capturing apparatus according to an embodiment of the present invention will now be described with reference to the accompanying drawings.

The radiographic capturing apparatus is an indirect radiographic capturing apparatus including a scintillator that converts incident radiation into light having different wavelengths, such as visible light, to generate electric signals. Alternatively, the radiographic capturing apparatus may be a direct radiographic capturing apparatus that directly detects incident radiation with the radiation detecting elements without a scintillator.

The radiographic capturing apparatus according to the present invention may be any radiographic capturing apparatus including a built-in power supply. In other words, the radiographic capturing apparatus according to the present invention is not limited to the portable radiographic capturing apparatus described below.

[Configuration of Radiographic Capturing Apparatus]

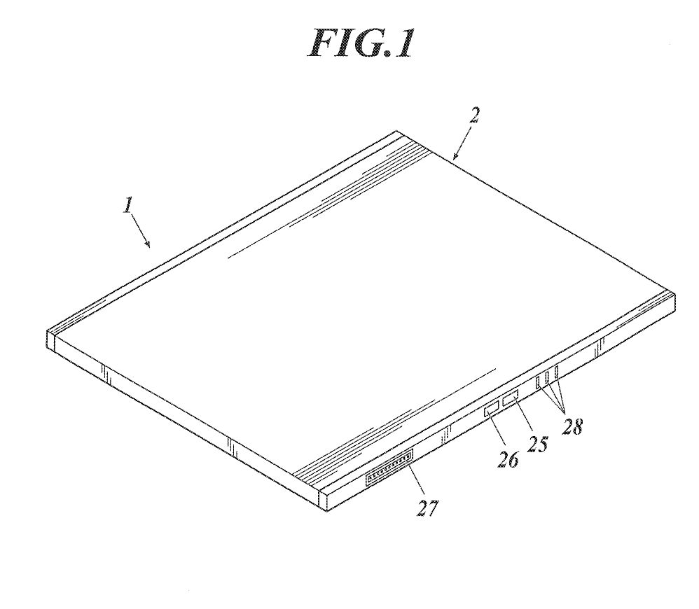

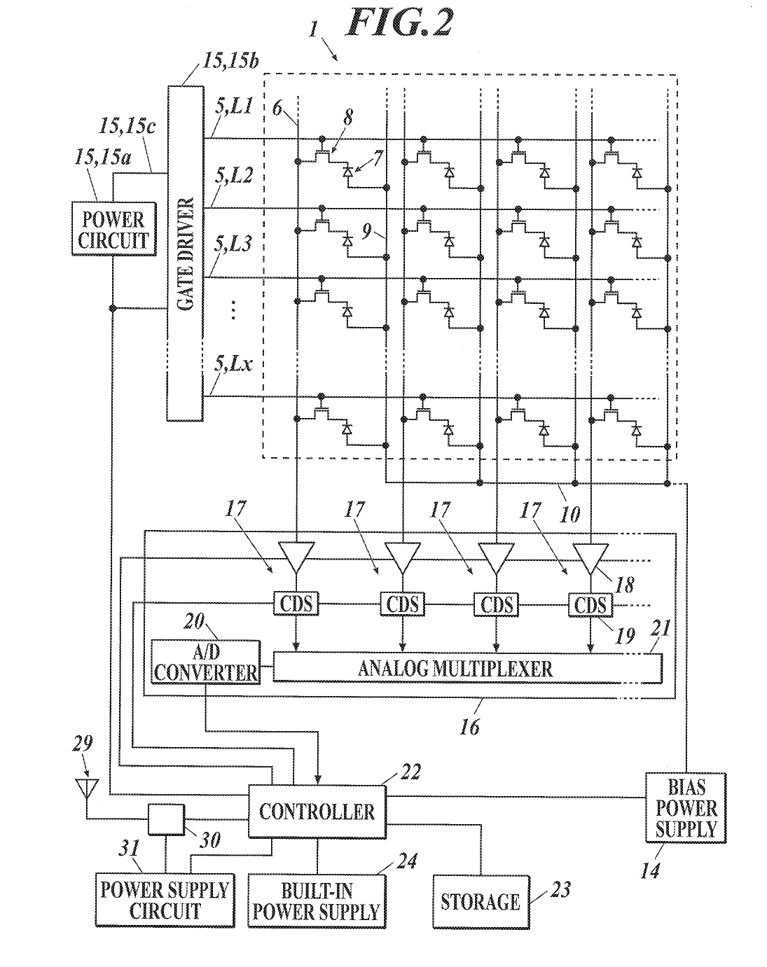

The configuration of the radiographic capturing apparatus 1 according to this embodiment will now be described. FIG. 1 is an external perspective view of the radiographic capturing apparatus 1. FIG. 2 is a block diagram illustrating an equivalent circuit of the radiographic capturing apparatus 1. The radiographic capturing apparatus 1 includes a two-dimensional array (matrix) of radiation detecting elements 7 (see FIG. 2) disposed on a sensor substrate (not shown) and accommodated in a casing 2 (see FIG. 1).

With reference to FIG. 1, the casing 2 of the radiographic capturing apparatus 1 is provided with a power switch 25, a selection switch 26, a connector 27, and an indicator 28 on its one side face. Although not illustrated, the casing 2 is provided with an antenna 29 (see FIG. 2) for wireless communication with external units on the opposite side face.

With reference to FIG. 2, the radiation detecting elements 7 are connected to bias lines 9. A reverse bias voltage is applied from a bias power supply 14 through the bias lines 9 and a connection line 10 to each radiation detecting element 7. The radiation detecting elements 7 are connected to respective switching elements or thin film transistors (TFTs) 8. The TFTs 8 are connected to signal lines 6. The radiation detecting elements 7 each generate charges in proportion to the doses of incident radiation.

In a scan driver 15, a power supply circuit 15a supplies ON and OFF voltages to a gate driver 15b via a line 15c. The gate driver 15b switches the received voltage and the switched voltage is applied to scanning lines 5 (L1) to 5(Lx). The TFTs 8 are turned off in response to an OFF voltage applied via the scanning lines 5 to disconnect the radiation detecting elements 7 and the respective signal lines 6 and cause accumulation of the electrical charges in the radiation detecting elements 7. The TFTs 8 are turned on in response to an ON voltage applied via the scanning lines 5 and cause the electrical charges accumulated in the radiation detecting elements 7 to be discharged via the signal lines 6.

The signal lines 6 are connected to respective reader circuits 17 in a reader IC 16. During the reading of image data D, an ON voltage is sequentially applied from the gate driver 15b to the scanning lines 5(L1) to 5(Lx). In response to turning-on of the TFTs 8, the charges in the radiation detecting elements 7 are discharged to the respective reader circuits 17 through the respective TFTs 8 and signal lines 6, and voltage values corresponding to the electrical charges are output from amplifier circuits 18.

Correlated double sampling circuits (“CDSs” in FIG. 2) 19 read the voltage values from the amplifier circuits 18 and output analog image data D. The image data D are sequentially sent to an A/D converter 20 via an analog multiplexer 21, converted to digital image data D at the A/D converter 20, and then stored in a storage 23.

A controller 22 includes a computer (not shown) provided with a central processing unit (CPU), a read only memory (ROM), a random access memory (RAM), and an input/output interface connected to a bus, and a field programmable gate array (FPGA). The controller 22 may be composed of a dedicated controller circuit.

The controller 22 is connected to the storage 23 provided with a static RAM (SRAM), a synchronous DRAM (SDRAM), and a NAND flash memory; a built-in power supply 24 including a lithium ion capacitor; and a wireless communication circuit or wireless communication device 30 that establishes wireless communication with external units via the antenna 29. The wireless communication circuit or wireless communication device 30 according to this embodiment establishes wireless communication in accordance with a wireless LAN scheme or a Bluetooth (registered trademark) scheme. Hereinafter, the wireless communication circuit or wireless communication device 30 is simply referred to as “wireless communication circuit 30.”

As described above, the controller 22 instructs the operation of the scan driver 15 and the reader circuits 17 to conduct the reading process of the image data D, stores the image data D to the storage 23, and transfers the image data D to an external unit via the wireless communication circuit 30.

[Configuration Involving Wireless Communication]

The wireless communication circuit 30 has two load states; a sleep state in which wireless communication cannot be established with an external unit but power consumption is significantly low and an awake state in which wireless communication can be established with power consumption higher than that in the sleep state.

Upon switching the load state, the wireless communication circuit 30 sends a state-switching signal Ss corresponding to load-state information indicating the switching of the load state (i.e., a signal indicating that the load state is to be switched to the sleep state or awake state) to the controller 22. In the following description, the wireless communication circuit 30 transmits a signal indicating the switching of the load state of the wireless communication circuit 30 (state-switching signal Ss) to the controller 22. Alternatively, the wireless communication circuit 30 may switch between high and low voltages to notify of the switching of the load state of the wireless communication circuit 30.

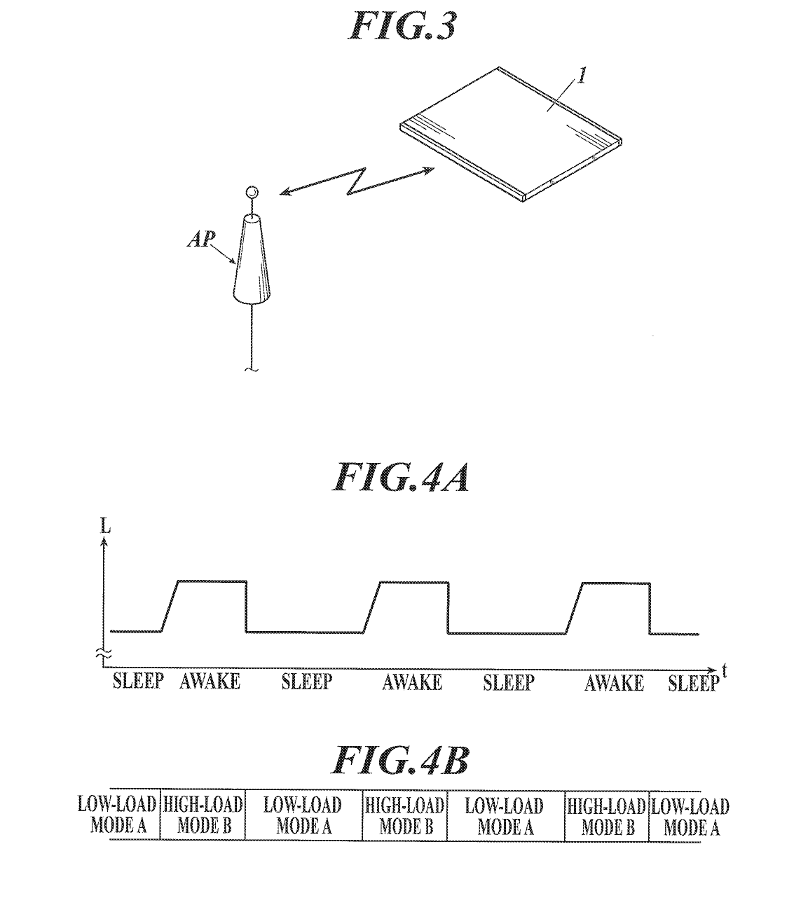

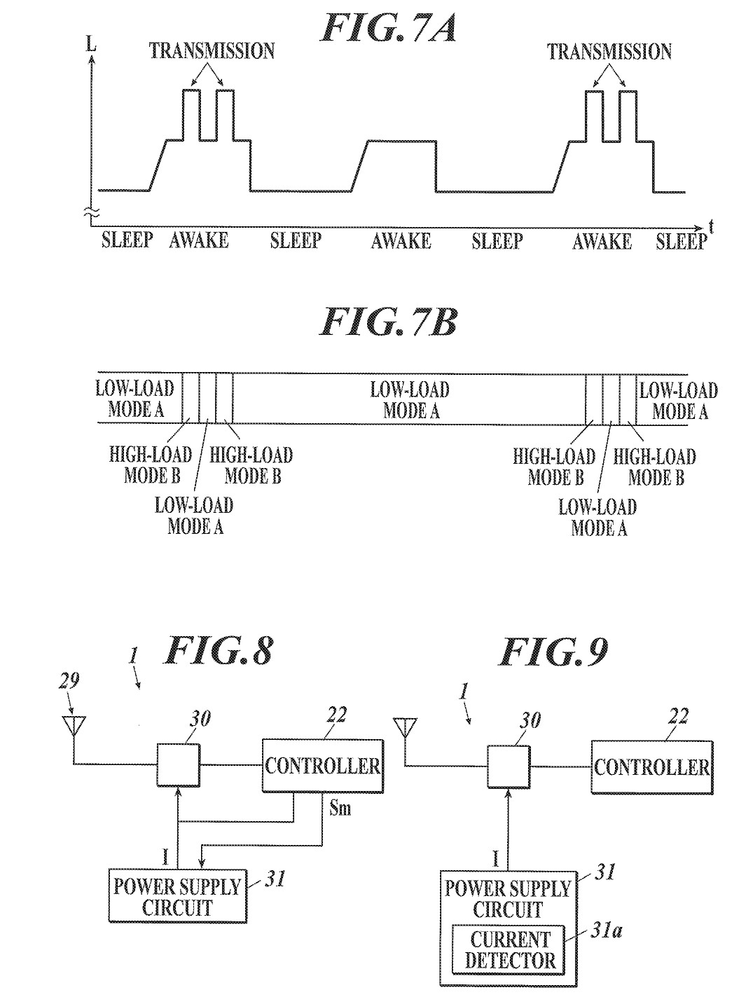

In this embodiment, an access point AP (see FIG. 3) transmits a beacon at a predetermined cycle. Before receiving a beacon from the access point AP, the wireless communication circuit 30 of the radiographic capturing apparatus 1 switches the load state from the sleep state to the awake state, as illustrated in FIG. 4A. Upon reception of the beacon from the access point AP, the wireless communication circuit 30 switches the load state from the awake state to the sleep state. In the graphs in FIGS. 4A and 7A, the top of graphs represents the highest load L.

The wireless communication circuit 30 according to this embodiment switches the load state between the sleep state and the awake state in a cycle in synchronization with the transmission cycle of beacons from the access point AP. The wireless communication circuit 30 sends a state-switching signal Ss (or a voltage, the same hereinafter) to the controller 22 every switching of the load state. The wireless communication circuit 30 is always in the awake state during image capturing with the radiographic capturing apparatus 1, for example.

The wireless communication circuit 30 is connected to a power supply circuit 31 that supplies electrical power to the wireless communication circuit 30. The power supply circuit 31 receives electrical power from the built-in power supply 24 (see FIG. 2). The power supply circuit 31 has at least two load modes: a low-load mode A and a high-load mode B. The power supply circuit 31 can switch between the low-load mode A and the high-load mode B.

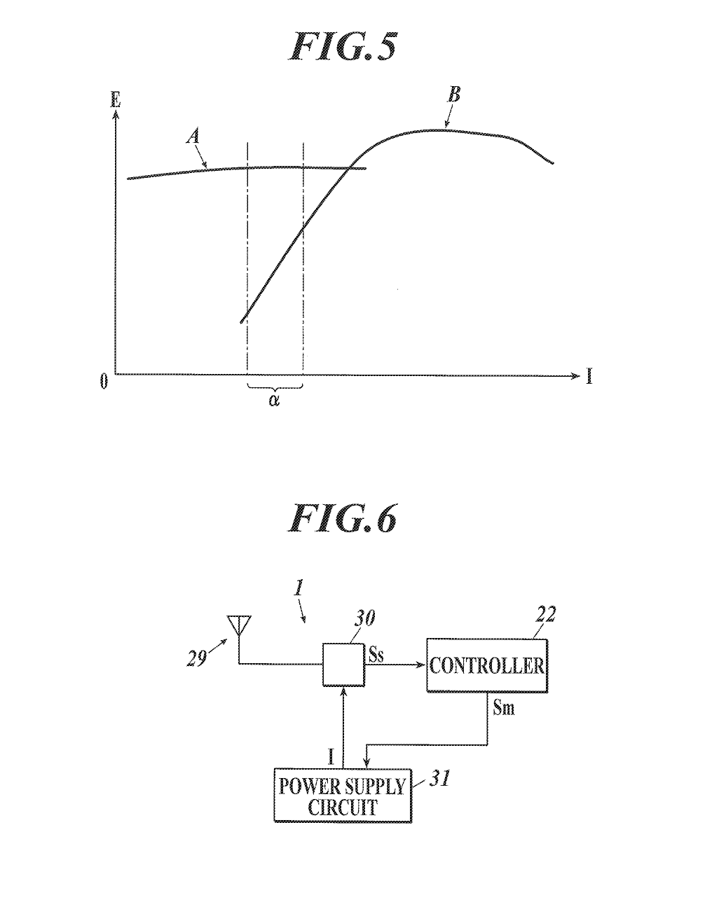

As illustrated in FIG. 5, the power supply circuit 31 in the low-load mode A according to this embodiment can supply only a current having a small value I to the wireless communication circuit 30, but the power supply efficiency E is high in the low-load mode A; whereas the power supply circuit 31 in the high-load mode B can supply a current having a value I larger than that in the low-load mode A, but the power supply efficiency E in the high-load mode B is lower than that in the low-load mode A during feeding of a current having a small value I (within the range a in FIG. 5, for example). In FIG. 5, the horizontal axis represents a current value I in a logarithmic scale.

The controller 22 according to this embodiment acquires load-state information of the wireless communication circuit 30 from the wireless communication circuit 30. Specifically, the controller 22 according to this embodiment acquires load-state information in the form of a state-switching signal Ss from the wireless communication circuit 30.

The controller 22 switches the load mode of the power supply circuit 31 on the basis of the load state of the wireless communication circuit 30. In detail, the controller 22 sends a mode-switching signal Sm to the power supply circuit 31 to switch the power supply circuit 31 to the high-load mode B upon detection of a first load state of the wireless communication circuit 30 and switch the power supply circuit 31 to the low-load mode A upon detection of a second load state of the wireless communication circuit 30. The first load state is available only in the high-load mode B of the power supply circuit 31, whereas the second load state is a load state other than the first load state. The controller 22 of the radiographic capturing apparatus 1 according to this embodiment functions as a switcher in the present invention.

The controller 22 functioning as a switcher will be described below. Instead of the controller 22, a circuit separate from the controller 22 may function as the switcher. In the description below, the power supply circuit 31 enters the low-load mode A in response to the sleep state of the wireless communication circuit 30 and supplies a current having a small value I from the power supply circuit 31 to the wireless communication circuit 30, whereas the power supply circuit 31 enters the high-load mode B in response to the awake state of the wireless communication circuit 30 and supplies a current having a large value I from the power supply circuit 31 to the wireless communication circuit 30.

In more specific, the following description is based on a case where the power supply circuit 31 in the low-load mode A can only supply a current having a value I smaller than 100 mA (in the high-load mode B, a current having a value I of 100 mA or more) and the wireless communication circuit 30 in the awake state requires a current having a value I of 100 mA or more.

[Operation]

The operation of the radiographic capturing apparatus 1 according to this embodiment will now be described. In an exemplary case, the wireless communication circuit 30 switches the load state to the awake state upon reception of a beacon from the access point AP, as illustrated in FIG. 4A, for example. At the same time, the wireless communication circuit 30 sends a state-switching signal Ss (indicating the switching to the awake state, in this case) to the controller 22, as illustrated in FIG. 6.

Upon reception of the state-switching signal Ss (i.e., the load-state information) from the wireless communication circuit 30, the controller 22 sends a mode-switching signal Sm to the power supply circuit 31 to switch the power supply circuit 31 to the high-load mode B, as illustrated in FIG. 4B, because the awake state of the wireless communication circuit 30 is available only in the high-load mode B of the power supply circuit 31.

In another exemplary case, the wireless communication circuit 30 switches to the sleep state upon reception of a beacon from the access point AP and sends a state-switching signal Ss (indicating the switching to the sleep state) to the controller 22. Upon reception of the state-switching signal Ss, the controller 22 sends a mode-switching signal Sm to the power supply circuit 31 to switch the power supply circuit 31 to the low-load mode A because the sleep state of the wireless communication circuit 30 is available also in the low-load mode A of the power supply circuit 31.

In this embodiment, the power supply circuit 31 certainly switches from the low-load mode A to the high-load mode B in response to the switching of the wireless communication circuit 30 from the sleep state to the awake state, as illustrated in FIGS. 4A and 4B. The power supply circuit 31 certainly switches from the high-load mode B to the low-load mode A in response to the switching of the wireless communication circuit 30 from the awake state to the sleep state.

Thus, when the wireless communication circuit 30 in the awake state receives a beacon from the access point AP, the power supply circuit 31 in the high-load mode B feeds a current having a large value I to the wireless communication circuit 30 at high power supply efficiency E, as illustrated in FIG. 5, to appropriately operate the wireless communication circuit 30. When the wireless communication circuit 30 is in the sleep state, the power supply circuit 31 in the low-load mode A feeds a current having a small value I at high power supply efficiency E, as illustrated in FIG. 5.

### Advantageous Effects

As described above, the switcher (controller 22) of the radiographic capturing apparatus 1 according to this embodiment certainly switches the load mode of the power supply circuit 31 for supplying electrical power to the wireless communication circuit or the wireless communication device 30 from the low-load mode A to the high-load mode B or vice versa in response to the switching of the load state of the wireless communication circuit or the wireless communication device 30.

Thus, electrical power can be fed efficiently from the power supply circuit 31 to the wireless communication circuit or the wireless communication device 30 at high power supply efficiency E in either the sleep state (low-load state) or the awake state (high-load state) of the wireless communication circuit or the wireless communication device 30. This leads to efficient use or low consumption of the power of the built-in power supply 24 (see FIG. 2) in the radiographic capturing apparatus 1.

This increases the operable time of the radiographic capturing apparatus 1 and the number of capturable images per charge of the built-in power supply 24. Thus, the radiographic capturing apparatus 1 can capture images with an increased efficiency.

In the embodiment described above, the wireless communication circuit 30 (or wireless communication device 30, the same hereinafter) in the awake state operates with a current having a large value I fed from the power supply circuit 31 in the high-load mode B. In another embodiment, the wireless communication circuit 30 in the awake state may operate with a current having a small value I fed from the power supply circuit 31 and receive a current having a large value I from the power supply circuit 31 only during a transmission process of a signal, etc.

For example, the situation is assumed where the power supply circuit 31 in the low-load mode A can feed a current having a value I smaller than 100 mA, and the wireless communication circuit 30 in the sleep state or the awake state operates with the current smaller than 100 mA but requires a current having a value I of several hundred milliamperes during a signal transmission process.

In this case, the wireless communication circuit 30 sends state-switching signals Ss (see FIG. 6) indicating the start and end of the transmission process in the wireless communication circuit 30 to the controller 22 at the start and end of the transmission process, respectively.

The controller 22 receives the state-switching signal Ss or load-state information from the wireless communication circuit 30. If the state-switching signal Ss indicates the start of the transmission process, the controller 22 sends a mode-switching signal Sm to the power supply circuit 31 to switch the power supply circuit 31 to the high-load mode B so that a current having a large value I required for the transmission process is fed from the power supply circuit 31 to the wireless communication circuit 30.

If the state-switching signal Ss indicates the end of the transmission process, the controller 22 sends a mode-switching signal Sm to the power supply circuit 31 to switch the power supply circuit 31 to the low-load mode A so that a current having a small value I sufficient for the operations other than the transmission process is fed from the power supply circuit 31 to the wireless communication circuit 30.

With reference to FIGS. 7A and 7B, the power supply circuit 31 is switched to the high-load mode B to supply a current having a large value I to the wireless communication circuit 30 at high power supply efficiency E (see FIG. 5), only during the transmission process at the wireless communication circuit 30. The power supply circuit 31 switches to the low-load mode A to supply a current having a small value I to the wireless communication circuit 30 at high power supply efficiency E, during any operation other than the transmission process.

In the radiographic capturing apparatus 1 according to this embodiment, the power supply circuit 31 can efficiently feed electrical power to the wireless communication circuit 30 at high power supply efficiency E during either the transmission process or any other operation carried out by the wireless communication circuit 30. This leads to efficient use or low consumption of the power of the built-in power supply 24 (see FIG. 2) in the radiographic capturing apparatus 1.

### First Modification

In the embodiment described above, the controller 22 or switcher sends a mode-switching signal Sm based on the state-switching signal Ss sent in response to the switching of the load state of the wireless communication circuit 30 (i.e., switching from the sleep state to the awake state or vice versa), to the power supply circuit 31 to switch the load mode (low-load mode A or high-load mode B) of the power supply circuit 31.

According to a first modification illustrated in FIG. 8, the controller 22 may detect the current having the value I fed from the power supply circuit 31 to the wireless communication circuit 30 without the state-switching signal Ss from the wireless communication circuit 30, detect the load state of the wireless communication circuit 30 on the basis of the detected current value I, and send a mode-switching signal Sm based on the detected load state to the power supply circuit 31, to switch the load mode of the power supply circuit 31.

In detail, an increase in load L of the wireless communication circuit 30 after switching of the wireless communication circuit 30 from the sleep state to the awake state, as illustrated in FIG. 4A, increases the value I of the current fed from the power supply circuit 31 to the wireless communication circuit 30, for example. The controller 22 or switcher may set a threshold Ith in advance and determine the wireless communication circuit 30 to be in the sleep state if the detected current value I is smaller than a predetermined threshold Ith and in the awake state if the detected current value I is larger than or equal to the threshold Ith.

Also in this first modification, the controller 22 certainly switches the load mode of the power supply circuit 31 from the low-load mode A to the high-load mode B (or vice versa) in response to switching of the wireless communication circuit 30 from the sleep state to the awake state (or vice versa), to feed a current from the power supply circuit 31 to the wireless communication circuit 30 at high power supply efficiency E, as in the embodiment illustrated in in FIG. 4B.

In the first modification, electrical power can be efficiently fed from the power supply circuit 31 to the wireless communication circuit 30 at high power supply efficiency E in either the sleep state or the awake state of the wireless communication circuit 30. This leads to efficient use or low consumption of the power of the built-in power supply 24 in the radiographic capturing apparatus 1. The same configuration can be applied to the cases illustrated in FIGS. 7A and 7B.

### Second Modification

In the first embodiment and first modification of the present invention, the controller 22 of the radiographic capturing apparatus 1 functions as a switcher. According to a second modification, a current detector 31a functioning as a switcher may be provided in the power supply circuit 31, as illustrated in FIG. 9.

In other words, the switcher is provided in the power supply circuit 31. In the second modification, the controller 22 of the radiographic capturing apparatus 1 is not involved in the switching of the load mode of the power supply circuit 31.

Similar to the controller 22 or the switcher according to the first modification, the current detector 31a of the power supply circuit 31 may compare a threshold Ith and the current value I fed from the power supply circuit 31 to the wireless communication circuit 30 and switch the load mode of the power supply circuit 31.

Also in the second modification, the power supply circuit 31 (i.e., the switcher or current detector 31a of the power supply circuit 31) certainly switches the load mode of the power supply circuit 31 from the low-load mode A to the high-load mode B (or vice versa) in response to switching of the wireless communication circuit 30 from the sleep state to the awake state (or vice versa), to feed a current from the power supply circuit 31 to the wireless communication circuit 30 at high power supply efficiency E, as in the embodiment illustrated in FIG. 4B.

Also in the second modification, electrical power can be efficiently fed from the power supply circuit 31 to the wireless communication circuit 30 at high power supply efficiency E in either the sleep state or the awake state of the wireless communication circuit 30. This leads to efficient use or low consumption of the power of the built-in power supply 24 in the radiographic capturing apparatus 1. The same configuration can be applied to the cases illustrated in FIGS. 7A and 7B.

### Third Modification

As described above, the wireless communication circuit 30 of the radiographic capturing apparatus 1 should respond to the beacons transmitted from the access point AP (see FIG. 3) at a predetermined cycle even in the sleep state. This causes the wireless communication circuit 30 to periodically enter the awake state to respond to the beacons, as illustrated in FIG. 4A.

Thus, the radiographic capturing apparatus 1 according to the first and second modifications may include a phase lock loop (PLL) that learns the cycle of switching of the load mode of the power supply circuit 31 or the cycle of transmission of mode-switching signals Sm to the power supply circuit 31 by the switcher (the controller 22 or the current detector 31a of the power supply circuit 31) during the sleep state of the wireless communication circuit 30.

Such configurations achieve the advantageous effects described above and autonomous control of switching the power supply circuit 31 from the low-load mode A to the high-load mode B immediately before the switching of the wireless communication circuit 30 from the sleep state to the awake state. Thus, electrical power can be smoothly and appropriately fed from the power supply circuit 31 to the wireless communication circuit 30 at high efficiency.

The embodiment and modifications described above should not be construed to be limited the present invention and may be appropriately modified without departing from the scope of the present invention.

1. A radiographic capturing apparatus comprising:
a two-dimensional array of a plurality of radiation detecting elements;
a wireless communication circuit or a wireless communication device which establishes wireless communication with an external unit; and
a power supply circuit which feeds electrical power to the wireless communication circuit or the wireless communication device and is switchable between a low-load mode and a high-load mode, the power supply circuit being able to feed a current having a larger value in the high-load mode than in the low-load mode, a power supply efficiency of the power supply circuit in the high-load mode being lower than the power supply efficiency of the power supply circuit in the low-load mode when a current having a small value as in the low-load mode is fed in the high-load mode;
a switcher which detects or obtains a load state of the wireless communication circuit or the wireless communication device and switches a load mode of the power supply circuit based on the detected or obtained load state of the wireless communication circuit or wireless communication device; and
a built-in power supply which feeds electrical power to the power supply circuit.

2. The radiographic capturing apparatus according to claim 1, wherein
the switcher detects or obtains a first load state and a second load state of the wireless communication circuit or wireless communication device, the first load state being available only in the high-load mode of the power supply circuit, the second load state being a state other than the first load state, and
the switcher switches the power supply circuit to the high-load mode upon detecting or obtaining the first load state of the wireless communication circuit or wireless communication device, and switches the power supply circuit to the low-load mode upon detecting or obtaining the second load state of the wireless communication circuit or wireless communication device.

3. The radiographic capturing apparatus according to claim 1, wherein the switcher is provided in the power supply circuit.

4. The radiographic capturing apparatus according to claim 1, wherein the switcher detects a value of a current fed from the power supply circuit to the wireless communication circuit or wireless communication device and detects the load state of the wireless communication circuit or the wireless communication device based on the detected current value.

5. The radiographic capturing apparatus according to claim 1, wherein the wireless communication circuit or wireless communication device establishes wireless communication in accordance with a wireless LAN scheme.

6. The radiographic capturing apparatus according to claim 1, wherein the wireless communication circuit or wireless communication device establishes wireless communication in accordance with a Bluetooth scheme.
